(19) United States
(12) Patent Application Publication
(10) Pub. No.: US 2002/0161066 A1
(43) Pub. Date: Oct. 31, 2002

(54) Method for making a nanofiltration membrane, and resulting membrane

(76) Inventors: Jean-Christophe Remigy, Cugnaux (FR); Philippe Aptel, Toulouse (FR); Sandrine Desclaux, Fonsorbes (FR); Alain Ricard, Toulouse (FR)

Correspondence Address: YOUNG & THOMPSON, 745 SOUTH 23RD STREET 2ND FLOOR, ARLINGTON, VA, 22202

(21) Appl. No.: 10/049,370

(22) PCT Filed: Jun. 5, 2001
(86) PCT No.: PCT/FR01/01725

(30) Foreign Application Priority Data
Jun. 14, 2000 (FR) 00 07549

(51) Int. Cl. C08G 2/00; C08F 2/46; C08J 3/28; C07C 1/00

(52) U.S. Cl. 522/6; 522/7

(57) ABSTRACT

The invention concerns a method for producing a nanofiltration membrane. At least one face (30) of a porous membrane (3) is put in the presence of a grafting composition comprising at least one monomer grafted by radical polymerisation and at least one crosslinking agent, that is however free from photoinitiator, and of light radiation capable of forming free radicals during a predetermined period adapted so as to obtain nanofiltration properties. The invention also extends to the nanofiltration membrane obtained in this way of which the properties may be precisely predetermined and which resist ageing.

The invention concerns the preparation of a nanofiltration membrane, in particular one having a degree of retention of ionic species that is not zero for a permeability greater than 10⁻⁶ l·h⁻¹·m⁻²·Pa⁻¹ and designed to receive a liquid to be filtered under a low feed pressure, in particular between 10⁵ and 10⁶ Pa (contrary to reverse osmosis membranes operating at a high pressure of several megapascals). Such a membrane may serve in particular for softening drinking water, for removing chemical pollutants from water or for separating low molecular weight organic molecules etc.

The production of such membranes is difficult. Indeed, contrary to ultrafiltration membranes (which do not hold back ionic inorganic species and have a permeability greater than 5.10⁻⁴ l·h⁻¹·m⁻²·Pa⁻¹) of which the production is now conventional and well controlled, it is not known how to control and provide precisely the filtration properties (degree of retention, cut-off threshold, permeability) of a nanofiltration membrane.

In particular, if it is desired to employ the known phase inversion technique for the production of ultrafiltration membranes, by adapting the parameters in order to obtain a nanofiltration membrane, the result is very random and variable according to the dexterity of the preparer and cannot be foreseen in the sense that it is not known what operations are needed to prepare a membrane with precisely determined filtration properties.

It is also known that it is possible to prepare a nanofiltration membrane from a symmetrical ultrafiltration membrane by the interfacial reaction of two grafting monomers disposed either side of the membrane so as to be in contact and to react by polycondensation in the region of the pores of the membrane. This technique requires the use of two reagents and is thus costly and complex to put into practice in as much as the contacting interface between the two grafting monomers must be positioned very precisely in the region of the pores that are to be partially obstructed. Moreover, although the polymer formed by polycondensation is embedded in the pores, it is not grafted onto the membrane, so that the nanofiltration membrane obtained is subject to not inconsiderable ageing and loses its nanofiltration properties with time.

In addition, the methods for producing nanofiltration membranes by chemical means are relatively polluting.

Many documents teach the grafting of a polymerizable grafting monomer, in particular by radical polymerization in the presence of UV radiation, on at least one face of an ultrafiltration membrane so as to obtain a surface with specific functional properties without modifying the size of the pores (hydrophilic, anti-blocking properties, etc.). For example, U.S. Pat. No. 5,468,390 describes a process for the photochemical grafting of polymerizable vinyl grafting monomers in the presence of free radicals on the surface of an ultrafiltration membrane of polyarylsulfone in the presence of UV radiation in the absence of an activator or free radical initiator, so as to give the membrane blocking resistance properties and so as to increase its permeability.

U.S. Pat. No. 4,618,533 also teaches the direct preparation of a coating of a hydrophilic crosslinked polymer formed of a grafting monomer polymerised in situ in the presence of a free radial initiator (photoinitiator) on the surface of a hydrophobic ultrafiltration membrane. This document explains that previous grafting techniques by polymerisation on the surface were difficult to put into practice since they resulted in the pores being blocked. With the method described in this document, the configuration of the pores and thus the filtration properties (cut-off threshold, permeability, etc.) remain unchanged.

U.S. Pat. No. 5,814,372 for which the applicant is the same as for U.S. Pat. No. 4,618,533, also describes a composite porous ultrafiltration membrane (pore size between 10 nm and 10 μm) formed by polymerisation of a self-crosslinking grafting monomer on the surface, under UV, in the presence of a photo initiator. Here again, the filtration properties remain unchanged. As explained in U.S. Pat. No. 5,814,372, the membrane of U.S. Pat. No. 4,618,533 is subject to ageing.

Many other documents describe similar processes modifying the functional properties of a microporous membrane by grafting crosslinked or uncrosslinked polymers, without changing the filtration properties (for example FR-2 688 418, U.S. Pat. No. 5,629,084, WO-9603202, etc.)

In addition, U.S. Pat. No. 5,256,503 or U.S. Pat. No. 5,425,865 teach the grafting of an acrylic polymer into the pores of a microporous membrane of polyethylene or polysulfone filled by impregnation under vacuum in the presence of a crosslinking agent and under UV radiation in the presence of a photoinitiator, so as to block the pores and so as to obtain a membrane impermeable to liquids but permeable to inorganic ionic species, that can serve for example as an ionic exchange membrane in electrochemical devices. Such membranes have properties that are exactly opposite to nanofiltration membranes, which must retain ions and must be permeable as much as possible to liquids.

As a result of this it has been considered up to now that grafting by radical polymerisation in situ under UV of a grafting monomer on the surface of an ultrafiltration membrane had the consequence either of modifying the functional properties of the surface without modifying the size of the pores (or even of increasing its permeability), or of producing blockage of the pores, destroying any filtration properties.

In this context, the object of the invention is to provide a method for preparing a nanofiltration membrane that is simple to put into practice, making it possible to obtain precise, predictable and reproducible nanofiltration properties which last over the period in which they are to be used, the membrane resisting ageing.

The object of the invention is also to provide such a method which is compatible with the technical and economic constraints of an industrial scale application, and which makes it possible to obtain a nanofiltration membrane at reduced cost and with respect for the environment

The object of the invention is also to provide a nanofiltration membrane having precise predetermined nanofiltration properties, that resists ageing and which is not costly.

More particularly, the object of the invention is to provide a nanofiltration membrane having a degree of retention of ionic inorganic species greater than 1%, in particular greater than 10%, for a permeability greater than 10⁻⁶ l·h⁻¹·m⁻²·Pa⁻¹.

To this end, the invention concerns a method for preparing a nanofiltration membrane, wherein:

the starting point is a porous membrane, a so-called supporting membrane, having at least one face, a so-called grafting face, having filtration properties in the range defined by microfiltration or ultrafiltration and, at least on this grafting face, at least one agent, a so-called photosensitive agent, capable of generating free radicals when it is subjected to light radiation,

the grafting face is put into the presence of:

a grafting composition containing at least one monomer, a so-called grafting monomer, capable of forming at least one polymer by radical polymerisation, and at least one crosslinking agent adapted so as to bring about crosslinking of at least one polymer formed by radical polymerisation, the molar quantity of crosslinking agent(s) being less than that of the grafting monomer(s) in the composition, this grafting composition being free from a photoinitiating agent,

light radiation capable of activating the formation of free radicals by the photosensitive agent of the support membrane in the absence of a photoinitiating agent in the grafting composition during a predetermined period as a function of the characteristics of the light radiation so as to obtain the nanofiltration properties of the membrane.

Accordingly, contrary to the general teaching of the prior art, the inventors have found that it is possible to modify in a permanent way (resistance to ageing) the filtration properties of a microporous or mesoporous membrane, in a controlled and predictable manner by carrying out grafting of a polymer by the radical polymerisation of grafting polymers in the presence of light radiation whilst, in combination, use is made of a membrane incorporating a photosensitive agent, and radical polymerisation is carried out in the presence of a cross-linking agent in the absence of a photoinitiator in the grafting composition, control of the period of irradiation making it possible, all other things being equal, (irradiation power, concentrations in the grafting composition, constitution of the supporting membrane) to obtain the desired nanofiltration properties without blocking the pores. The polymer is simultaneously formed, grafted and crosslinked on the grafting face. This photochemical process without a photoinitiator minimises the number of chemical compounds used, which makes it possible to reduce the cost of the reagents in the final cost of the nanofiltration membrane and makes its exploitation on the industrial scale possible with due respect for the environment.

Accordingly, contrary to U.S. Pat. No. 5,468,390, which does not employ a crosslinking agent, the membrane obtained according to the invention resists ageing and has a modified pore size. Contrary to U.S. Pat. No. 4,618,533 or U.S. Pat. No. 5,814,372 which provide for the presence of a photoinitiator, the invention excludes any photoinitiator and this simple fact makes it possible to modify the filtration properties while obtaining high ageing resistance. There is no clear and definitive explanation for this surprising result. One of the possible explanations could be that the presence of a photoinitiator harms grafting and ageing resistance on account of the fact that it encourages the joint or previous formation of crosslinked free homopolymers or copolymers within the grafting composition, whereas in a process according to the invention, free radicals (and thus jointly polymerisation, crosslinking and grafting) only form on the surface of the porous membrane.

Advantageously and according to the invention, the supporting membrane is a mesoporous ultrafiltration membrane with a permeability of between 5.10⁻⁴ and 10⁻² l·h⁻¹·m⁻²·Pa⁻¹, in particular between 10⁻³ and 6.10⁻³ l·h⁻¹·m⁻²·Pa⁻¹. It is also possible to start from a microporous microfiltration membrane, but the treatment time will be longer. The geometry of the supporting membrane (as well as that of the nanofiltration membrane according to the invention obtained from this supporting membrane) may be of any geometry whatsoever. It may consist in particular of a membrane, a so-called flat membrane, or a membrane in the form of a hollow fibre or of a bundle of hollow fibres. In the case of hollow fibres, the external cylindrical face of the hollow fibre may generally be treated so as to present nanofiltration properties.

In addition, advantageously and according to the invention, the supporting membrane includes at least one photosensitive agent chosen from the group formed of polysulfones and their derivatives—in particular polysulfone, (polymethysulfone), polyarylsulfones and polyethersulfone—aromatic polyketones, polyphenylene oxides, aromatic polyimides, polyetherketones, copolymers and mixtures of polymers containing at least one photosensitive agent chosen from the group formed of polysulfones or their derivatives, aromatic polyketones, polyphenylene oxides, aromatic polyimides and polyetherketones.

The polysulfones are polymers of the formula:

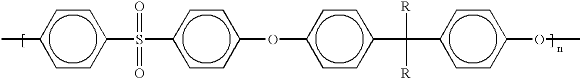

R representing any organic group.

If R is an alkyl, the polymer is a polyalkylsulfone. The polymer known under the name of “polysulfone” (without any other details) corresponds to polymethylsulfone (R being a methyl).

When R is an aryl, the polymer is a polyarylsulfone, for example polyphenylsulfone.

The polyethersulfone derivative is the polymer of formula:

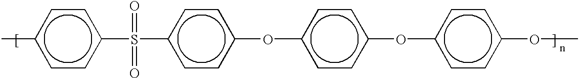

Advantageously and according to the invention, the supporting membrane consists substantially of at least one photosensitive polymer and this polymer is advantageously chosen from the group mentioned above. This being the case, it is sufficient for the grafting face to have one such photosensitive agent.

Accordingly, as a variant, the membrane may be formed substantially of a matrix of a non-photosensitive material, which incorporates at least one photosensitive agent (photoinitiator) distinct from this non-photosensitive material.

Each grafting monomer of the grafting composition must be chosen so as to be compatible with radical polymerisation under light irradiation and so as to be able to form a grafted crosslinked polymer in a covalent manner on the grafting face of the supporting membrane. In addition, the crosslinking agent must be compatible with the polymer obtained so as to produce crosslinking simultaneously with the polymerisation and grafting. Advantageously and according to the invention, the grafting composition contains at least one grafting monomer containing in its formula at least one unsaturated covalent bond, in particular at least one carbon-carbon double bond, and at least one crosslinking agent including in its formula at least two unsaturated covalent bonds, in particular at least two carbon-carbon double bonds.

Advantageously and according to the invention, the grafting composition contains at least one vinyl grafting monomer. More particularly, advantageously and according to the invention, the grafting composition contains at least one grafting monomer chosen from the group comprising acrylic acid; acrylamide; methacrylic acid and their acrylate, methacrylate and acrylamide derivatives; vinyl pyridines and their alkyl or carbazole derivatives; maleic anhydride; vinyl acetate; vinyl sulfonic acid; vinyl phosphoric acid; 4-styrene sulfonic acid; N-vinyl pyrrolidone. In particular, these different grafting monomers are compatible with the group of polymers mentioned above forming the photosensitive agent of the supporting membrane.

Accordingly, as grafting monomers that can be used advantageously according to the invention, reference may be made in particular to : acrylic acid; acrylamide; methacrylic acid; ethyl methacrylate; ethyl acrylate; hydroxyalkyl acrylates, in particular 1-hydroxy prop-2-yl acrylate, 2-hydroxyprop-1-yl acrylate, 2,3-dihydroxypropyl acrylate, 2-hydroxyethyl acrylate, 2-hydroxypropyl acrylate, 3-hydroxypropyl acrylate, 2,3-dihydroxypropyl acrylate; hydroxyalkyl methacrylates such as 2-hydroxyethyl methacrylate; N-monomethyl acrylamide; N-dimethylacrylamide; 2-vinylpyridine; 2-methyl-5-vinylpyridine, 2-vinyl-5-ethylpyridine and N-vinyl carbazole.

In addition, advantageously and according to the invention, the grafting composition contains at least one crosslinking agent chosen from the group of acrylates, methacrylates and difunctional acrylamides, namely those including at least two carbon-carbon double bonds (divinyl or, more generally, dialkene). As a crosslinking agent that can be advantageously chosen in a method according to the invention, reference may be made to the following compounds:

triallyl isocyanurate; triallyl cyanurate; 1,5-hexadiene-3-ol; 2,5-dimethyl-1,5-hexadiene; 1,5-hexadiene; 1,7-octadiene; 3,7-dimethyl-2,6-octadiene-1-ol; divinylbenzene; tetraethylene glycol diacrylate; polyethylene glycol dimethacrylate; methylene bisacrylamide.

Advantageously and according to the invention, the supporting membrane and the grafting monomer(s) of the grafting composition are chosen so that the photosensitive agent(s) have an absorption spectrum in a wavelength region where the grafting monomer(s), and preferably also the crosslinking agent, have substantially no absorption, and light radiation is chosen that does not emit outside this range.

In addition, advantageously and according to the invention, light radiation is applied with a wavelength or wavelengths situated outside the absorption spectrum of the grafting monomer(s) of the grafting composition.

In this way, light radiation brings about the formation of free radicals by the photosensitive agent(s) on the surface of the supporting membrane, but not the formation of free radicals by the grafting monomers and/or by the crosslinking agent that can induce synthesis of the polymers (homopolymers or copolymers) within the grafting composition. In particular, in the case where acrylic acid is used as the grafting monomer of the grafting composition, advantageously and according to the invention, light radiation is applied with a wavelength or wavelengths greater than 300 nm. Indeed, acrylic acid has an absorption spectrum situated below 300 nm, whereas sulfone has an absorption spectrum of between 300 and 330 nm. To this end, advantageously and according to the invention, an ultraviolet lamp is used to apply the light radiation that is surrounded by a glass tube (in particular forming an outer cooling circuit) capable of filtering out wavelengths below 300 nm, made in particular of DURAN 50®.

Advantageously and according to the invention, light radiation is applied with a wavelength or wavelengths of between 200 and 600 nm, so as to deliver light energy of between 0.1 J/cm² and 300 J/cm², preferably between 0.7 and 160 J/cm². For light radiation with a wavelength or wavelengths of between 300 nm and 600 nm, the light energy should advantageously lie between 0.1 J/cm² and 200 J/cm², preferably between 0.5 J/cm² and 100 J/cm².

The grafting composition may be an aqueous or non-aqueous solution (in an organic solvent) or may be formed of a grafting monomer or grafting monomers and a crosslinking agent or crosslinking agents in the liquid state (without a solvent).

The concentrations of grafting monomer(s) and crosslinking agent(s) may vary according to the nature and reactivity of these compounds, the photosensitive agent and the supporting membrane. Advantageously and according to the invention, the grafting composition contains between 1% and 10% by mass, in particular of the order of 2.5% by mass, of grafting monomer(s). In addition, advantageously and according to the invention, the grafting composition includes a quantity of crosslinking agent(s) of between 0.1 molar % and 10 molar % of the quantity of grafting monomer(s).

Advantageously and according to the invention, in order to put the grafting face in the presence of the grafting composition, the supporting membrane is immersed in a bath of the grafting composition in the form of a deoxygenated liquid solution, preferably at least substantially free from polymerisation inhibitor. It should be noted that in practice a certain quantity of inhibitor does not impede polymerisation under the conditions of the invention. The solution must be deoxygenated so as to prevent any reaction between oxygen and free radicals.

The method of the invention may be put into practice either discontinuously or continuously. In both cases, two variants are possible. In a first variant according to the invention, light radiation is applied while the grafting face is immersed in a bath of the grafting composition. In a second variant according to the invention, the grafting face is immersed in a bath of the grafting composition and is then removed from this bath and light radiation is then applied.

The invention extends to a nanofiltration membrane obtained by a method according to the invention.

The invention thus concerns a nanofiltration membrane characterised in that it comprises :

a porous membrane, a so-called supporting membrane, having at least one face, a so-called grafting face, having filtration properties in the field defined by microfiltration and ultrafiltration and,

a graft of at least one crosslinked polymer grafted onto the grafting face, this graft being adapted so as to confer nanofiltration properties on the grafting face. The term “graft” denotes a quantity of a polymer or polymers bonded in a covalent manner to the grafting face.

Advantageously and according to the invention, the membrane has a degree of retention of ionic inorganic species greater than 10% for a permeability greater than 10⁻⁶ l·h⁻¹·m⁻²·Pa⁻¹ and substantially retains its properties with time and in use.

Advantageously and according to the invention, the supporting membrane is a microporous or mesoporous membrane consisting substantially of at least one polymer chosen from the group formed of polysulfones and their derivatives, in particular polysulfone (polymethylsulfone), polyarylsulfones and polyethersulfone, aromatic polyketones, polyphenylene oxides, aromatic polyimides, polyetherketones, copolymers and mixtures of polymers containing at least one photosensitive agent chosen from the group formed of polysulfones or their derivatives, aromatic polyketones, polyphenylene oxides, aromatic polyimides and polyetherketones.

Advantageously and according to the invention, the graft of a crosslinked polymer or polymers is formed of at least one vinyl polymer, in particular at least one polyacrylic polymer.

The membrane according to the invention may be symmetrical if all its outer faces are treated as grafting faces, or asymmetrical in the contrary case. In addition, nothing will prevent the starting point from being a supporting membrane that is itself asymmetrical.

Advantageously and according to the invention, the membrane is in the form of a hollow fibre.

The invention extends to a method for producing a membrane according to the invention, as well as a membrane and a method of production characterised in combination by all or part of the features mentioned above or hereinafter.

Other objectives, advantages and features of the invention will become apparent on reading the following examples and description which refer to the accompanying figures in which:

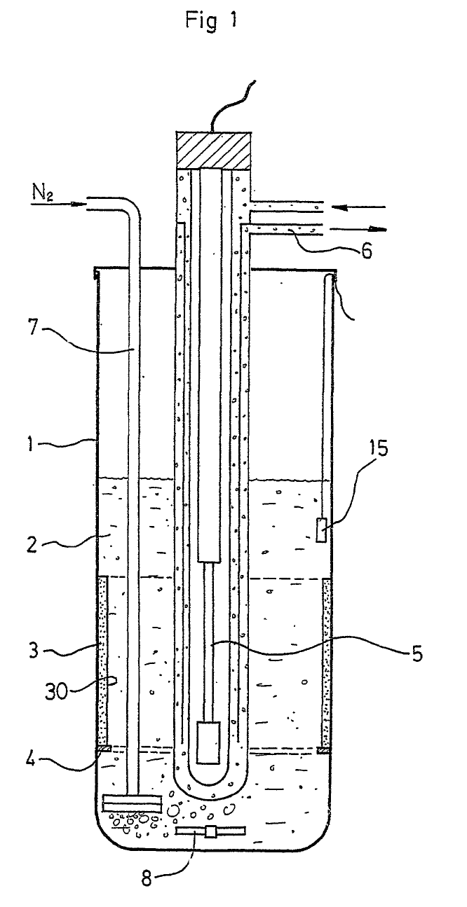

FIG. 1 is a diagram illustrating an axial section of an installation for implementing a discontinuous method according to the invention,

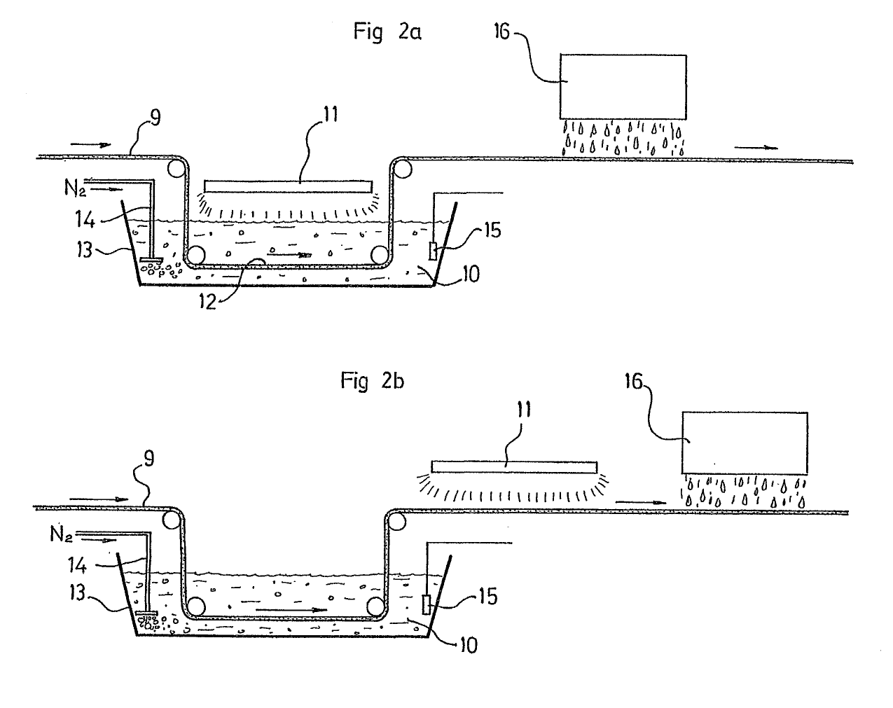

FIGS. 2a and FIG. 2b are diagrams illustrating respectively variants of the implementation of a continuous method according to the invention,

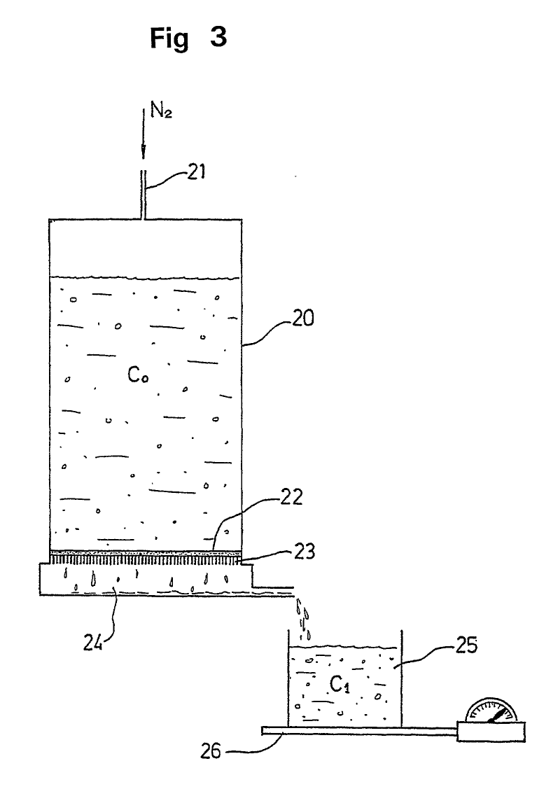

FIG. 3 is a diagram illustrating the principle for measuring the degree of ionic retention of a membrane according to the invention.

The installation shown in FIG. 1 enables a method for producing a nanofiltration membrane according to the invention to be implemented in a discontinuous manner. This installation comprises a cylindrical vessel 1 receiving a grafting solution 2. A microporous microfiltration or mesoporous ultrafiltration supporting membrane 3 with a rectangular shape is immersed in the solution 2, rolled up on itself and pressed against the wall of the vessel 1, and held upright for example with the aid of a ring 4. An ultraviolet lamp 5 is immersed axially (along the axis of symmetry of the vessel 1 so as to be equidistant from the different zones of the inner face 30 of the membrane 3) within the liquid solution 2 so as to emit opposite the grafting face 30 of the supporting membrane 3. This ultraviolet lamp 5 is preferably a quartz lamp cooled by a water circuit 6 formed of a DURAN 50® glass tube. A conduit 7 enables gaseous nitrogen to be bubbled into the bottom of the vessel 1 so as to deoxygenate the solution 2. A stirrer 8 is advantageously provided at the bottom of the vessel 1.

In order to produce a nanofiltration membrane made of polysulfone, the starting point is a microporous or mesoporous membrane made of polysulfone and a grafting composition is used containing at least one grafting monomer such as acrylic acid and at least one crosslinking agent such as methylene bisacrylamide, in suitable concentrations. The membrane 3 is placed in the vessel 1, the lamp 5 being left outside the bath. Nitrogen is bubbled into the solution 2 with stirring until the concentration of dissolved oxygen falls to a value of 0.25 mg/l of oxygen. This dissolved oxygen concentration may be measured with the aid of a conventional probe 15. When this threshold concentration is reached, the lamp 5 is immersed in the bath facing the membrane. The lamp 5 is illuminated sufficiently in advance before being immersed in the bath so that it reaches its permanent operating state. The membrane is then irradiated for a predetermined period and is then withdrawn from the reactor and washed with distilled water.

FIG. 2a illustrates an installation for carrying out a similar process continuously, the supporting membrane 3 being formed of a continuous strip 9 moving forward in a bath 10 of liquid grafting solution. A source of ultraviolet radiation 11 is positioned above the bath 10 opposite the grafting face 12 of the membrane 9 to be treated. The bath 10 is formed in a vessel 13 which also receives a conduit 14 for bubbling gaseous nitrogen into the bath 10.

The variant of FIG. 2b differs from the preceding one in that the source of light radiation 11 is applied downstream from the bath 10 after the membrane 9 has been removed from the bath 10. This is in actual fact impregnated with the grafting liquid solution and application of the radiation is sufficient to bring about polymerisation and crosslinking of the polymer, at the same time as it is grafted on the face 12 of the membrane.

In the case of hollow fibres, the light source 11 may be formed of one or more cylindrical ultraviolet ovens through which the fibre passes axially so as to ensure uniform peripheral illumination.

The rate of passage of the membrane 9 is adapted according to the desired duration for the application of radiation onto the grafting face 12, with a view to obtaining a nanofiltration membrane.

In continuous installations, a water-washing unit 16 is also provided downstream from the treatment with ultraviolet radiation. Nevertheless, it should be noted that no drying step is necessary.

The treatment temperature enabling nanofiltration membranes to be obtained according to the invention is ambient temperature. In practice, this temperature may vary between 10° C. and 50° C.

The installation in FIG. 1 was used to prepare examples 1 and 2 described below. For these examples, a mesoporous ultrafiltration supporting membrane 3 was used made of polysulfone marketed by the POLYMEM company, with a hydraulic permeability equal to 10^{-3±10}⁻⁴ l·h⁻¹m⁻²·Pa⁻¹, and initially not offering any retention of ionic inorganic species. The supporting membrane 3 was placed at a distance of the order of 16 mm from the lamp 5, and the irradiated area was approximately 100 cm². The volume of the liquid grafting solution was 650 cm³ and the lamp 5 was a Hanau Heraeus TQ 150 ultraviolet lamp of which the cooling tube was made of DURAN 50® glass. This lamp 5 emitted at different wavelengths according to table 1 below.

TABLE 1

| | |
|---|---|
| Wavelength | +HL,8 |

| (nm) | 254 | 313 | 366 | 436 | 546 |
|---|---|---|---|---|---|
| Light flux | 0.0 | 2.5 | 5.8 | 3.6 | 4.6 |
| with glass | | | | | |
| tube (W) | | | | | |

The grafting solution was an aqueous solution of acrylic acid (as a monomer) and methylene bisacrylamide, as the crosslinking agent.

In order to measure hydraulic permeabilities, a cylindrical cell is used (for example AMICON 8050®) with a capacity of 50 cm³, and with an internal diameter of 43 mm. The working area of the membrane was 13.2 cm². The hydraulic permeability was measured with the aid of osmosis-purified water.

FIG. 3 represents the installation used for measuring the degree of retention of ionic inorganic species (or the degree of ionic retention). This degree of ionic retention was measured with reference to the calcium ion Ca²⁺ with the aid of a synthetic solution of calcium chloride containing 50 mg/l of calcium ions prepared with osmosis/purified water. The installation comprised a reservoir 20 pressurised by nitrogen introduced into the upper part under a pressure of 4.10⁵ Pa through a conduit 21. The solution was placed in the reservoir 20 which was closed at its lower part by the membrane 22 to be tested, placed above a mesh 23 which emerged into a collector 24 collecting the liquid passing through the membrane 22 and the mesh 23. The assembly formed the cylindrical cell. The collector 24 poured the collected solution (permeate) into a vessel 25 placed on an electronic balance 26 for determining the mass of permeate collected as a function of time. This measurement made it possible to calculate the hydraulic permeability by dividing the mass of permeate by the filtering area, pressure and the measurement time. The concentrations of calcium C₀ of the initial solution placed in the vessel 20, and C₁ of the permeate, were measured with the aid of a plasma torch. The concentration C₁ of the permeate was measured for a predetermined volume factor (ratio of the initial volume of the solution in the vessel over its final volume), in particular equal to 15. The degree of ionic retention Tr was calculated according to the following equation:

Tr=1−C₁/C₀

### EXAMPLE 1

In this example, a concentration of 2.5% (by mass) of acrylic acid was used in the grafting solution. The concentration of methylene bisacrylamide was 0.0267% (by mass) in the solution, corresponding to approximately 1.25 molar % of the quantity of acrylic acid. In this example, the ultraviolet irradiation time was held fixed at 5 min. Nevertheless, the permeability of the supporting membrane 3 was varied. The following table indicated the results obtained concerning the permeability of the modified nanofiltration membrane as well as its degree of calcium retention.

TABLE 2

| | | Degree of calcium |
|---|---|---|
| | Permeability of | retention |
| Permeability of | membrane modified | (synthetic |
| supporting | according to the | solution |
| membrane | invention | containing 50 mg/l |
| (l.h⁻¹.m⁻².Pa⁻¹) | (l.h⁻¹.m⁻².Pa⁻¹) | of Ca²⁺ |
| 10⁻³ | 0.38.10⁻⁵ | 57% |
| 5.10⁻³ | 0.8.10⁻⁵ | 25% |
| 6.10⁻³ | 1.4.10⁻⁵ | 23% |

### EXAMPLE 2

In this example, the mass concentration of acrylic acid in the solution was varied as well as the mass concentration of the crosslinking agent. A comparative example was also carried out using a solution of acrylic acid in the presence of a photoinitiator (benzoin) and without the crosslinking agent. The ultraviolet irradiation time was varied from 3.5 to 7 min.

Ageing of the membrane was performed by soaking it in osmosis-purified water at 60° C. for a predetermined period. A test was also carried out on a nanofiltration membrane according to the invention using, for measuring the degree of calcium retention, not a solution made with osmosis-purified water, but one made with tap water having a concentration of 31.3 mg/l of calcium ions.

The following table 3 gives the results obtained:

TABLE 3

| | | Methylene | | | | | |
|---|---|---|---|---|---|---|---|
| | | bis- | | | | | |
| | Acrylic | acrylamide | | | | | Degree of |
| | acid | conc.(mass | Benzoin | | | | Ca |
| | conc. | % of | conc. (mass | | | | retention |
| | (mass % | solution) | % of | | | | (Ca |
| | of | Cross- | solution) | | Ageing time, | | solution |
| | solution) | linking | Photo- | Irradiation | days (water | Permeability | with 50 |
| | Monomer | agent | initiator | time (mm) | 60° C.) | (l/h/m²/bar | mg/l Ca²⁺) |
| Support | 0 | 0 | 0 | 0 | 0 | 100 | 0% |
| membrane | | | | | | | |
| Conventional | 2.5 | 0 | 0.1 | 5 | 0 | 0.94 | 8% |
| membrane + | | | | | | | |
| photoinitiator | | | | | | | |
| Aged convent- | 2.5 | 0 | 0.1 | 5 | 7 | 13.46 | 0% |
| ional | | | | | | | |
| membrane + | | | | | | | |
| photoinitiator | | | | | | | |
| Present | 2.5 | 0.027 | 0 | 3 | 0 | 0.87 | 18% |
| invention | | | | | | | |
| Present | 2.5 | 0.027 | 0 | 5 | 0 | 0.38 | 57% |
| invention | | | | | | | |
| Present | 2.5 | 0.027 | 0 | 7 | 0 | 0.34 | 85% |
| invention | | | | | | | |
| Present | 1.0 | 0.0021 | 0 | 5 | 0 | 2.4 | 4% |
| invention | | | | | | | |
| Present | 7.0 | 0.015 | 0 | 5 | 0 | 0.16 | 18% |
| invention | | | | | | | |
| Present | 2.5 | 0.027 | 0 | 3 | 0 | 0.85 | 15% |
| invention | | | | | | | |
| Present | 2.5 | 0.0535 | 0 | 3 | 0 | 0.47 | 14% |
| invention | | | | | | | |
| Present | 2.5 | 0.027 | 0 | 3 | 7 | 0.86 | 17% |
| invention, aged | | | | | | | |
| Present | 2.5 | 0.027 | 0 | 5 | 7 | 0.38 | 56% |
| invention, aged | | | | | | | |
| Present | 2.5 | 0.027 | 0 | 7 | 7 | 0.34 | 86% |
| invention, aged | | | | | | | |
| Present | 2.5 | 0.027 | 0 | 5 | 0 | 0.3 | 74% |
| invention, (tap | | | | | | | |
| waterwith 31.3 | | | | | | | |
| mg/l Ca²⁺ | | | | | | | |

The first three lines of table 3 show comparative examples. As can be seen, the fact of using a photoinitiator in the absence of a crosslinking agent considerably reduces, on the one hand, the final degree of calcium retention obtained, but above all destroys any resistance-to-ageing property of the membrane. On the contrary, by means of the invention, the membrane obtained not only has a high degree of calcium retention with satisfactory permeability, but above all these properties are maintained after ageing for seven days with water at 60° C.

The membranes used in this example were prepared discontinuously as mentioned above with the installation of FIG. 1. The first test mentioned in table 3 (concentrations of acrylic acid of 2.5% by mass and methylene bisacrylamide of 0.023% by mass, 3 min irradiation without ageing) was repeated by soaking for 15 min in a deoxygenated solution in the absence of ultraviolet, and then by removing the grafting solution from the vessel 1 before inserting the UV lamp. This test was thus representative of the continuous process in which the membrane was extracted from the bath before being irradiated by ultraviolet. The same results were obtained as those mentioned in table 3.

### EXAMPLE 3

In this example, a hollow nanofiltration fibre was produced continuously with an installation similar to that represented in FIG. 2b.

The starting point was a hollow ultrafitration fibre made of polysulfone rolled on a reel which was passed continuously through a bath of grafting composition degassed with nitrogen, identical to that of example 1, and then in two Hoenle FOZFR 250® UV ovens, 290 nm<λ<600 nm, mounted in series, and then in a three-cylinder device known as a “tricylinder”, providing a constant rate of progression. The fibre was then washed with osmosis-purified water. The following table 4 gives the results obtained.

TABLE 4

| | | | | |
|---|---|---|---|---|
| Irradiation time (s) | 0 | 2 | 3 | 6 |
| Permeability | 100 | 22 | 7 | 5 |
| (l.h⁻¹.m⁻².bar⁻¹) | | | | |
| Degree of calcium | 0% | 2.5% | 6% | 16% |
| retention, | | | | |
| Synthetic solution of | | | | |
| calcium chloride with | | | | |
| 50 mg/l Ca²⁺ | | | | |

It will be seen that a nanofiltration membrane was obtained in the form of a hollow fibre with a short treatment time, in a simple and economical manner, that could be carried out on the industrial scale.

The invention may be the subject of many alternative methods of implementation with respect to the embodiments and examples mentioned above.

1. A method for preparing a nanofiltration membrane, wherein:
the starting point is a porous membrane (3,9), a so-called supporting membrane, having at least one face, a so-called grafting face (12,30), having filtration properties in the range defined by microfiltration or ultrafiltration and, at least on this grafting face, at least one agent, a so-called photosensitive agent, capable of generating free radicals when it is subjected to light radiation,
the grafting face (12,30) is put in the presence of:
a grafting composition containing at least one monomer, a so-called grafting monomer, capable of forming at least one polymer by radical polymerisation, and at least one crosslinking agent adapted so as to bring about crosslinking of at least one polymer formed by radical polymerisation, the molar quantity of crosslinking agent(s) being less than that of the grafting monomer(s) in the composition, this grafting composition being free from a photoinitiating agent,
light radiation capable of activating the formation of free radicals by the photosensitive agent of the supporting membrane (3,9) in the absence of a photoinitiating agent in the grafting composition, during a predetermined period, adapted as a function of the characteristics of the light radiation so as to obtain the nanofiltration properties of the membrane.

2. The method as claimed in claim 1, wherein the supporting membrane (3,9) is a mesoporous ultrafiltration membrane with a permeability of between 5.10⁻⁴ and 10⁻² l·h⁻¹·m⁻²·Pa⁻¹, in particular between 10⁻³ and 6.10⁻³ l·h⁻¹·m⁻³·Pa⁻¹.

3. The method as claimed in either of claims 1 or 2, wherein the supporting membrane (3,9) contains at least one photosensitive agent chosen from the group formed of polysulfones and their derivatives—in particular polysulfone, (polymethysulfone), polyaryl sulfones and polyethersulfone—aromatic polyketones, polyphenylene oxides, aromatic polyimides, polyetherketones, and copolymers and mixtures of polymers containing at least one photosensitive agent chosen from the group formed of polysulfones or of their derivatives, aromatic polyketones, polyphenylene oxides, aromatic polyimides and polyetherketones.

4. The method as claimed in one of claims 1 to 3, wherein the supporting membrane (3,9) consists substantially of at least one photosensitive polymer.

5. The method as claimed in claim 4, wherein the photosensitive polymer is chosen from the group formed of polysulfones and their derivatives—in particular polysulfone, (polymethysulfone), polyaryl sulfones and polyethersulfone—aromatic polyketones, polyphenylene oxides, aromatic polyimides, polyetherketones, and copolymers and mixtures of polymers containing at least one photosensitive agent chosen from the group formed of polysulfones or of their derivatives, aromatic polyketones, polyphenylene oxides, aromatic polyimides and polyetherketones.

6. The method as claimed in one of claims 1 to 5, wherein the grafting composition contains at least one grafting monomer containing in its formula at least one unsaturated covalent bond, in particular at least one carbon-carbon double bond, and at least one crosslinking agent including in its formula at least two unsaturated covalent bonds, in particular at least two carbon-carbon double bonds.

7. The method as claimed in one of claims 1 to 6, wherein the grafting composition contains at least one vinyl grafting monomer.

8. The method as claimed in one of claims 1 to 7, wherein the grafting composition contains at least one grafting monomer chosen from the group comprising acrylic acid; acrylamide; methacrylic acid and their acrylate, methacrylate and acrylamide derivatives; vinyl pyridines and their alkyl or carbazole derivatives; maleic anhydride; vinyl acetates; vinyl sulfonic acid; vinyl phosphoric acid; 4-styrene sulfonic acid; N-vinyl pyrolidone.

9. The method as claimed in one of claims 1 to 8, wherein the grafting composition includes at least one crosslinking agent chosen from the group of acrylates, methacrylates and difunctional acrylamides.

10. The method as claimed in claim 9, wherein the grafting composition includes at least one crosslinking agent chosen from the following group of compounds: triallyl isocyanurate; triallyl cyanurate; 1,5-hexadiene-3-ol; 2,5-dimethyl-1,5-hexadiene; 1,5-hexadiene; 1,7-octadiene; 3,7-dimethyl-2,6-octadiene-1-ol; divinylbenzene; tetraethylene glycol diacrylate; polyethylene glycol dimethacrylate; methylene bisacrylamide.

11. The method as claimed in one of claims 1 to 10, wherein light radiation is applied with a wavelength or wavelengths of between 200 nm and 600 nm, so as to deliver light energy of between 0.1 J/cm² and 300 J/cm², preferably between 0.7 and 160 J/cm².

12. The method as claimed in one of claims 1 to 11, wherein the supporting membrane (3,9) and the grafting monomer(s) of the grafting composition are chosen such that the photosensitive agent(s) has/have an absorption spectrum in a wavelength region where the grafting monomer(s) has/have substantially no absorption, and light radiation is chosen that does not emit outside this region.

13. The method as claimed in one of claims 1 to 12, wherein light radiation is applied with a wavelength or wavelengths situated outside the absorption region of the grafting monomer(s) of the grafting composition.

14. The method as claimed in one of claims 1 to 13, wherein light radiation is applied with a wavelength or wavelengths above 300 nm.

15. The method as claimed in one of claims 1 to 14, wherein an ultraviolet lamp (5,11) is used surrounded by a glass tube capable of filtering out wavelengths below 300 nm.

16. The method as claimed in one of claims 1 to 15, wherein the grafting composition contains between 1% and 10% by mass, in particular of the order of 2.5% by mass, of a grafting monomer or monomers.

17. The method as claimed in one of claims 1 to 16, wherein the grafting composition contains a quantity of crosslinking agent(s) of between 0,1 molar % and 10 molar % of the quantity of grafting monomer.

18. The method as claimed in one of claims 1 to 17 wherein, in order to put the grafting face (12,30) into the presence of the grafting composition, the supporting membrane (3,9) is immersed in a bath (2,10) of the grafting composition in the form of a deoxygenated liquid solution.

19. The method as claimed in one of claims 1 to 18, wherein light radiation is applied while the grafting face (12, 30) is immersed in a bath (2, 10) of grafting composition.

20. The method as claimed in one of claims 1 to 19, wherein the grafting face (12, 30) is immersed in a bath of the grafting composition, is then withdrawn from this bath and light radiation is then applied.

21. A nanofiltration membrane wherein it comprises:
a porous membrane (3,9), a so-called supporting membrane, having at least one face, a so-called grafting face (12,30) having filtration properties in the range defined by microfiltration and ultrafiltration,
a graft of at least one crosslinked polymer grafted onto the grafting face, this graft being adapted so as to confer nanofiltration properties on the grafting face.

22. The membrane as claimed in claim 21, wherein it has a degree of retention of ionic inorganic species greater than 10% for a permeability greater than 10⁻⁶ l·h⁻¹·m⁻²·Pa⁻¹, and retains, at least substantially, these properties with time and during use.

23. The membrane according to either of claims 21 or 22, wherein the supporting membrane (3,9) is a microporous or mesoporous membrane consisting substantially of at least one polymer chosen from the group formed of polysulfones and their derivatives—in particular polysulfone (polymethylsulfone), polyarylsulfones and polyethersulfone—aromatic polyketones, polyphenylene oxides, aromatic polyimides, polyetherketones, and copolymers and mixtures of polymers containing at least one photosensitive agent chosen from the group formed of polysulfones or their derivatives, aromatic polyketones, polyphenylene oxides, aromatic polyimides and polyetherketones.

24. The membrane as claimed in one of claims 21 to 23, wherein the graft of a crosslinked polymer or polymers is formed of at least one vinyl polymer.

25. The membrane as claimed in claim 24, wherein the graft of a crosslinked polymer or polymers is formed of at least one polyacrylic polymer.

26. The membrane as claimed in one of claims 21 to 25, wherein it is in the form of a hollow fibre.
